(19) United States
(12) Patent Application Publication
(10) Pub. No.: US 2008/0125368 A1
(43) Pub. Date: May 29, 2008

(54) METHODS

(75) Inventor: Frederick Wai Keung Tam, London (GB)

Correspondence Address: NIKOLAI & MERSEREAU, P.A., 900 SECOND AVENUE SOUTH, SUITE 820, MINNEAPOLIS, MN, 55402, US

(73) Assignee: Imperial Innovations Limited, London (GB)

(21) Appl. No.: 11/924,034

(22) Filed: Oct. 25, 2007

Related U.S. Application Data
(60) Provisional application No. 60/855,712, filed on Oct. 31, 2006.

(51) Int. Cl. A61K 38/00; C12Q 1/68; G01N 33/53

(52) U.S. Cl. 514/12; 435/6; 435/7.1

(57) ABSTRACT

A method for aiding in the assessment of peritoneal membrane injury or complications of peritoneal dialysis in a patient, the method comprising the step of determining the level of CCL18 and/or angiogenin in a sample from the patient. The method may be for assessing the likely progression of peritoneal membrane injury or complications of peritoneal dialysis in the patient or for assessing the likely progression of response of the patient to treatment. The peritoneal membrane injury may be a complication of peritoneal dialysis (PD).

The present invention relates to diagnosis and treatment of complications of peritoneal dialysis (PD).

### BACKGROUND

Inflammation and fibrosis are important disease mechanisms in the pathogenesis of long term complications for patients receiving peritoneal dialysis. Most of the previous research focuses on mediators that may cause either inflammation or fibrosis. Little attention has been given to find mediators that may be involved in both processes.

PD has the advantage of a more mobile life style enabling patients to work and travel, and is effective even when patients have poor vascular access for haemodialysis. With improved management of bacterial peritonitis, and use of automated PD (APD), patients are able to remain on PD for many years. However, development of ultrafiltration failure (UFF), and encapsulating peritoneal sclerosis (EPS) have now become major limitations ¹. A significant proportion of these patients need to switch over to haemodialysis. EPS is an uncommon but potentially fatal complication in long term PD patients, in which diffuse fibrosis of the peritoneum results in intestinal obstruction. Surgical treatment of EPS has been rarely successful. The efficacy of corticosteroid or tamoxifen treatment currently is not certain and most patients with EPS need to stop PD, experiencing recurrent episodes of intestinal obstruction. Some of them eventually rely on total parenteral nutrition. With increasing numbers of patients on long term PD, EPS has become a more frequent long term complication.

Long-term exposure to high dialysate glucose concentrations, bioincompatible dialysates and previous episodes of bacterial peritonitis, collectively appear to be the important clinical factors for development of UFF and EPS ^{2;3}. The likely mechanisms include chronic inflammation, neoangiogenesis and fibrosis ⁴. Pro-inflammatory cytokines, such as IL-1, TNF-α, IL-6 and MCP-1, are important in peritoneal membrane injury during and also after bacterial infection ^{5;6}. IL-6 and VEGF are associated with inflammation and neoangiogenesis in PD patients ⁷. Most of the evidence for profibrotic factors (e.g. TGF-β1 and CTGF) in the pathogenesis of peritoneal membrane injury has come from the use of mesothelial cell cultures and rodent models. However, the clinical correlations of these cytokines and growth factors with UFF and EPS are still not established ⁸. Therefore, robust mechanistic markers for staging and/or prognosis of PD complications are still greatly needed.

### DESCRIPTION OF THE INVENTION

We have identified two mediators from the study of peritoneal dialysis patients, CCL18 and angiogenin, which are amongst mediators important in inflammation and fibrosis, and neoangiogenesis respectively. Involvement of these mediators has not previously been reported in patients receiving dialysis. Measurement of CCL18 and angiogenin in biological fluids provides quantitative data on disease activity, and provides biomarkers to monitor patients' response to treatment. This also provides a biomarker to assess the possibility of new treatment, such as new dialysis fluid, in causing complications. Furthermore, treatments that suppress production of CCL18 and/or angiogenin, or their downstream effects, may be useful in prevention or treatment of complications of peritoneal dialysis.

A first aspect of the invention provides a method for aiding in the assessment of complications of peritoneal dialysis in a patient, the method comprising the step of determining the level of CCL18 and/or angiogenin nucleic acid or protein in a sample from the patient.

CCL18 is described in, for example, reference 11. Angiogenin is described in, for example, reference 15, as discussed in Example 1.

The sample may be, for example, a blood serum sample or a blood plasma sample or a sample of spent peritoneal dialysis fluid. Spent peritoneal dialysis fluid contains cytokines released from the patient, making it suitable for such analysis. In addition, in the spent dialysate there are cell sediments (mainly macrophages and mesothelial cells) with the consequence that measurement of mRNA in such samples is feasible. In the blood, there are peripheral blood monocytes, so measurement of mRNA for CCL18 and angiogenin may provide indications of synthesis of CCL18 and angiogenin. Furthermore, studying of DNA polymorphism (of the coding DNA sequence or promoter region) may provide additional information about individual patients who are genetically prone to produce too much or too little of CCL18 or angiogenin.

Using spent peritoneal dialysis fluid may provide the most useful result, as such a sample may be closest to the organ of injury (i.e. peritoneum). Using a blood serum or blood plasma sample may be more convenient in at least some circumstances or for some patients, and may therefore also be useful.

The method may be useful for aiding in the diagnosis of complications of peritoneal dialysis in a patient. The method may also or alternatively be useful for aiding in the assessment of the likelihood or likely severity or likely progression of complications of peritoneal dialysis in a patient. Thus, the method may be useful in predicting future response of the patient to PD. It may be useful in prognosis or aiding prognosis. The method may be used as an adjunct to known prognostic methods such as considerations of membrane permeability, glucose exposure, history (duration of PD, previous peritonitis, type of dialysis fluid).

The method may comprise the steps of (i) obtaining a sample containing nucleic acid and/or protein from the patient; and (ii) determining whether the sample contains a level of CCL18 or angiogenin nucleic acid or protein associated, with a complication of PD.

It will be appreciated that determining whether the sample contains a level of CCL18 or angiogenin nucleic acid or protein associated with a complication of PD may in itself be diagnostic (or prognostic) of a complication of PD or it may be used by the clinician as an aid in reaching a diagnosis or prognosis.

Thus, measurement of CCL18 or angiogenin levels may be performed or considered alongside other measurements or factors, for example membrane permeability, glucose exposure, history (duration of PD, previous peritonitis, type of dialysis fluid).

It will be appreciated that determining whether the sample contains a level of CCL18 or angiogenin nucleic acid or protein associated with a complication of PD may in itself be diagnostic (or prognostic) of a complication of PD or it may be used by the clinician as an aid in reaching a diagnosis or prognosis. The clinician may wish to take in to account these or other factors, as well as consider the level of CCL18 or angiogenin levels, before making a diagnosis. Measurement of CCL18 or angiogenin levels may provide more detailed information on the severity of individual disease mechanisms. With specific results from dialysate CCL18 and angiogenin, the contribution of inflammation/fibrosis vs neoangiogenesis may be assessed independently.

It will be appreciated that determination of the level of CCL18 or angiogenin in the sample will be useful to the clinician in determining how to manage peritoneal dialysis in the patient. For example, since elevated levels of CCL18 or angiogenin are associated with complications of PD, the clinician may use the information concerning the levels of CCL18 or angiogenin to facilitate decision making regarding treatment of the patient. Thus, if the level of CCL18 or angiogenin is indicative of a low likelihood of complications or low severity of complications or low likelihood of progression, unnecessary restriction of the PD may be avoided. Thus, without CCL18 or angiogenin measurement, the clinician may want to ask a long term PD patients (for example more than, 5 years of PD treatment) to change to haemodialysis to reduce the risk of complications. However, measurement of CCL18 and/or angiogenin may permit the risk to be assessed more accurately. Similarly, if the level of CCL18 or angiogenin is indicative of a high likelihood of complications or high severity of complications or high likelihood of progression, changes to the PD regime may be preferred. Thus, a more biocompatible dialysate may be prescribed. Other modalities of renal replacement therapy (transplantation and haemodialysis) may be planned earlier.

The level of CCL18 or angiogenin which is indicative of complications of PD may be defined as the increased level present in samples from patients with a known complication of PD over patients without a known complication of PD. The level of said CCL18 or angiogenin protein may be, for example, at least 1.1, 1.2 or 1½ fold higher in a sample from a patient with a known complication of PD, or it may be at least 2-fold or 3-fold higher. The level of mRNA encoding CCL18 or angiogenin may be, for example, at least 1½ fold higher in a sample from a patient with a known complication of PD, or it may be at least 2-fold or 3-fold higher, or at least 10, 50, 100, 500 or 1000-fold higher.

Determining the level of CCL18 and/or angiogenin in a sample from the patient may be determined using any suitable protein detection or quantitation method, for example using methods employing respectively antibodies specific for CCL18 or angiogenin. Thus, immunoassay techniques, preferably quantitative techniques, may be used, for example an antibody array or captured ELISA technique, for example as described in the Examples. Preferred embodiments relating to methods for detecting CCL18 or angiogenin protein includes enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference.

It will be appreciated that other antibody-like molecules may be used in the method of the inventions including, for example, antibody fragments or derivatives which retain their antigen-binding sites, synthetic antibody-like molecules such as single-chain Fv fragments (ScFv) and domain antibodies (dAbs), and other molecules with antibody-like antigen binding motifs.

Bioassays may alternatively be used for measuring CCL18 or angiogenin activity. For example, CCL18 stimulates fibroblast to produce collagen in vitro. Angiogenin stimulates DNA synthesis and proliferation of endothelial cells in culture. (reference 13) Ref: Proc Natl Acad Sci USA. 1997 Mar. 18; 94(6):2204-9. A putative angiogenin receptor in angiogenin-responsive human endothelial cells. Hu G F, Riordan J F, Vallee B L.

In one preferred embodiment of the invention it is determined whether the level of CCL18 or angiogenin nucleic acid, in particular mRNA, is a level associated with a complication of PD or peritoneal membrane injury. Preferably, the sample contains nucleic acid, such as mRNA, and the level of CCL18 or angiogenin is measured by contacting said nucleic acid with a nucleic acid which hybridises selectively respectively to CCL18 or angiogenin nucleic acid.

By “selectively hybridising” is meant that the nucleic acid has sufficient nucleotide sequence similarity with the said human nucleic acid that it can hybridise under moderately or highly stringent conditions. As is well known in the art, the stringency of nucleic acid hybridization depends on factors such as length of nucleic acid over which hybridisation occurs, degree of identity of the hybridizing sequences and on factors such as temperature, ionic strength and CG or AT content of the sequence. Thus, any nucleic acid that is capable of selectively hybridising as said is useful in the practice of the invention.

Nucleic acids which can selectively hybridise to the said human nucleic acid include nucleic acids which have >95% sequence identity, preferably those with >98%, more preferably those with >99% sequence identity, over at least a portion of the nucleic acid with the said human nucleic acid. As is well known, human genes usually contain introns such that, for example, a mRNA or cDNA derived from a gene would not match perfectly along its entire length with the said human genomic DNA but would nevertheless be a nucleic acid capable of selectively hybridising to the said human DNA. Thus, the invention specifically includes nucleic acids which selectively hybridise to CCL18 or angiogenin mRNA or cDNA but may not hybridise to a CCL18 or angiogenin gene. For example, nucleic acids which span the intron-exon boundaries of the CCL18 or angiogenin gene may not be able to selectively hybridise to the CCL18 or angiogenin mRNA or cDNA.

Typical moderately or highly stringent hybridisation conditions which lead to selective hybridisation are known in the art, for example those described in Molecular Cloning, a laboratory manual, 2nd edition, Sambrook et al (eds), Cold Spring Harbor Laboratory Press, Cold Spring Harbor, N.Y., USA, incorporated herein by reference.

An example of a typical hybridisation solution when a nucleic acid is immobilised on a nylon membrane and the probe nucleic acid is 500 bases or base pairs is:

### 6×SSC (saline Na⁺ citrate)

### 0.5% Na⁺ dodecyl sulphate (SDS)

100 μg/ml denatured, fragmented salmon sperm DNA

The hybridisation is performed at 68⁵⁸ C. The nylon membrane, with the nucleic acid immobilised, may be washed at 68° C. in 1×SSC or, for high stringency, 0.1×SSC.

20×SSC may be prepared in the following way. Dissolve 175.3 g of NaCl and 88.2 g of Na⁺ citrate in 800 ml of H₂O. Adjust the pH to 7.0 with a few drops of a 10 N solution of NaOH. Adjust the volume to 1 litre with H₂O. Dispense into aliquots. Sterilize by autoclaving.

An example of a typical hybridisation solution when a nucleic acid is immobilised on a nylon membrane and the probe is an oligonucleotide of between 15 and 50 bases is:

### 3.0 M trimethylammonium chloride (TMACl)

### 0.01 M Na⁺ phosphate (pH 6.8)

1 mm EDTA (pH 7.6)

### 0.5% SDS

100 μg/ml denatured, fragmented salmon sperm DNA
0.1% nonfat dried milk

The optimal temperature for hybridization is usually chosen to be 5° C. below the Tᵢ for the given chain length. Tᵢ is the irreversible melting temperature of the hybrid formed between the probe and its target sequence. Jacobs et al (1988) Nucl. Acids Res. 16, 4637 discusses the determination of Tᵢs. The recommended hybridization temperature for 17-mers in 3 M TMACl is 48-50^{□}C; for 19-mers, it is 55-57^{□}C; and for 20-mers, it is 58-66^{□}C.

By “nucleic acid which selectively hybridises” is also included nucleic acids which will amplify DNA from the said CCL18 or angiogenin mRNA by any of the well known amplification systems such as those described in more detail below, in particular the polymerase chain reaction (PCR). Suitable conditions for PCR amplification include amplification in a suitable 1× amplification buffer:

10× amplification buffer is 500 mM KCl; 100 mM Tris.Cl (pH 8.3 at room temperature); 15 mM MgCl₂; 0.1% gelatin.

A suitable denaturing agent or procedure (such as heating to 95° C.) is used in order to separate the strands of double-stranded DNA.

Suitably, the annealing part of the amplification is between 37^{□}C and 60^{□}C, preferably 50^{□}C.

Although the nucleic acid which is useful in the methods of the invention may be RNA or DNA, DNA is preferred. Although the nucleic acid that is useful in the methods of the invention may be double-stranded or single-stranded, single-stranded nucleic acid is preferred under some circumstances such as in nucleic acid amplification reactions.

The nucleic acid that is useful in the methods of the invention may be any suitable size. However, for certain diagnostic, probing or amplifying purposes, it is preferred if the nucleic acid has fewer than 10000, more preferably fewer than 1000, more preferably still from 10 to 100, and in further preference from 15 to 30 base pairs (if the nucleic acid is double-stranded) or bases (if the nucleic acid is single stranded). As is described more fully below, single-stranded DNA primers, suitable for use in a polymerase chain reaction, are particularly preferred.

The nucleic acid for use in the methods of the invention is a nucleic acid capable of hybridising to the CCL18 or angiogenin mRNA. Fragments of the CCL18 or angiogenin genes and cDNAs derivable from the mRNA encoded by the CCL18 or angiogenin genes are also preferred nucleic acids for use in the methods of the invention.

It is particularly preferred if the nucleic acid for use in the methods of the invention is an oligonucleotide primer which can be used to amplify a portion of the CCL18 or angiogenin nucleic acid, particularly CCL18 or angiogenin mRNA.

MCP-1 (well known in the art and see also reference 6) may be measured in addition to CCL18 and/or angiogenin. By combining these measurements, there may be increased specificity of the assessment.

The method may be useful for assessing response to PD in the patient.

The complication of peritoneal dialysis (PD) may be, for example, peritoneal membrane injury, ultrafiltration failure (UFF) or encapsulating peritoneal sclerosis (EPS). UFF and EPS may be consequences of peritoneal membrane injury.

Dialysate CCL18 and angiogenin are considered to be non-invasive markers of peritoneal membrane injury. In patients with high dialysate CCL18, a more biocompatible dialysate may be prescribed. In patients with high dialysate CCL18 and/or angiogenin, other modalities of renal replacement therapy (transplantation and haemodialysis) may be planned earlier.

A further aspect of the invention provides a method for assessing a peritoneal dialysis treatment regime or treatment regime for peritoneal membrane injury, the method comprising the step of determining the level of CCL18 and/or angiogenin protein or nucleic acid in a sample from patients receiving the treatment regime. The sample type is typically of the type discussed above in relation to the first aspect of the invention, for example blood serum or peritoneal dialysate. The method may, for example, be used to provide information on the likelihood of side effects of peritoneal dialysis in assessing the suitability of a proposed peritoneal dialysis solution. Thus, levels of CCL18 and/or angiogenin may be used as surrogate markers in clinical trials of a proposed peritoneal dialysis solution.

This can be important for new development of treatment and prevention of complications. Measurement of CCL18 and angiogenin provide the overall assessment of how multiple factors (including glucose exposure and history) integrate in causing pathological changes to the peritoneal membrane in the patients.

A further aspect of the invention provides a method for identifying a compound useful in peritoneal dialysis, for example in treating or preventing a complication of PD, or in treating or preventing peritoneal membrane injury the method comprising the steps of a) determining whether a test compound is capable of suppressing production of, or activity of, CCL18 or angiogenin in peritoneal tissue or a sample from a patient with peritoneal membrane injury or receiving PD, for example a patient with a complication of PD, and b) selecting a compound which is capable of suppressing production of; or activity of, CCL18 or angiogenin in peritoneal tissue or in a patient with peritoneal membrane injury or receiving PD, for example a patient with a complication of PD.

The method may comprise the step of determining whether a test compound is capable of suppressing production of, or activity of, CCL18 or angiogenin in a sample, for example a blood serum sample or peritoneal dialysate sample, from a patient receiving PD, for example a patient with a complication of PD, as discussed above.

In all aspects of the invention including the present aspect, the patient is typically a human. CCL18 has not been identified in rodents yet, so an animal model may not be suitable. An in vitro model may be most appropriate. Examples are: (1) Primary culture of peritoneal cells from spent dialysis fluid of patients; and (2) macrophage cell line.

A further aspect of the invention provides a method of treating a patient with peritoneal membrane injury or receiving PD, the method comprising the step of administering to the patient an effective amount of an antagonist of CCL18 and/or an antagonist of angiogenin. By antagonist of CCL18 or antagonist of angiogenin is meant a compound which suppresses production of, or activity of CCL18 or angiogenin, for example as determined by a method according to the preceding aspect of the invention.

The ability of CCL18 to induce collagen synthesis depends on activation of protein kinase C alpha, ERK2, phosphorylation of transcription factor Sp1 and basal expression of Smad3 in lung fibroblast in cell culture study. See: Luzina I. G. et al (2006) PKCalpha mediates CCL18-mediates CCL18-stimulated collagen production in pulmonary fibroblast. Am J. Resp. Cell Mol biology 35:298-305; Luzina I. G. et al (2006) CCL18-stimulated upregulation of collagen production in lung fibroblasts requires Sp1 signalling and basal Smad3 activity. J. Cell Physiol 206:221-228. Therefore, it is; likely that inhibition of these pathways may inhibit production of CCL18 in peritoneal dialysis patients.

DNA aptamers have been used, to inhibit action of angiogenin in cell proliferation assay in vitro. Ref. Nobile V et al (1998) Inhibition of human angiogenin by DNA aptamers: nuclear colocalization of an angiogenin-inhibitor complex. Biochemistry 37:6857-6863. The action of angiogenin may also be inhibited by neomycin (ref: Hu G F. Neomycin inhibits angiogenin-induced angiogenesis. Proc Natl Acad Sci USA. 1998 Aug. 18; 95:9791-5) and ERK 1/2 inhibitors (Ref Liu S, Yu D, Xu Z P, Riordan J F, Hu G F. Angiogenin activates Erk 1/2 in human umbilical vein endothelial cells. Biochem Biophys Res Commun. 2001 Sep. 14; 287:305-10).

The invention will now be described in more detail by reference to the following, non-limiting, Figures and Examples.

Any documents referred to herein are hereby incorporated by reference.

### FIGURE LEGENDS

FIG. 1. Autoradiograph showing antibody arrays of cytokines and growth factors in spent dialysate from a long term PD patients. High concentrations of CCL18 and angiogenin were detected. These concentrations were even higher than that of MCP-1 in the same samples.

FIG. 2. Dialysate CCL18 correlated positively with membrane permeability in PD patients.

FIG. 3. Dialysate CCL18 increased with duration on PD.

FIG. 4. Dialysate CCL18 increased with increasing dialysate glucose exposure.

FIG. 5. Dialysate CCL18 was higher with bioincompatible fluid (Dianeal).

FIG. 6. Dialysate CCL18 was higher in patients with previous peritonitis.

FIG. 7. Dialysate angiogenin correlated positively with membrane permeability in PD patients.

FIG. 8. Dialysate angiogenin increased with increasing dialysate glucose exposure.

FIG. 9. Quantity of Dialysate CCL18 correlated positively with membrane permeability in PD patients.

FIG. 10. Quantity of Dialysate CCL18 increased with duration on PD.

FIG. 11. Quantity of Dialysate CCL18 increased with increasing dialysate glucose exposure.

FIG. 12. Quantity of Dialysate CCL18 was higher with bioincompatible fluid (Dianeal).

FIG. 13. Quantity of Dialysate angiogenin correlated positively with membrane permeability in PD patients.

FIG. 14. Quantity of Dialysate Angiogenin higher in long term PD patients

FIG. 15. One year outcome of patients in CCL18 study.

FIG. 16. Serum CCL18 concentrations in one year follow-up. (A) Baseline serum CCL18 concentrations were higher in patients who switched to haemodialysis because of PD technique failure during follow-up. (B) Baseline serum, CCL18 concentrations were higher in patients who progressed to EPS group during follow-up.

FIG. 17. Dialysate CCL18 levels in one-year follow-up. (A). Baseline dialysate CCL18 levels were higher in patients who switched to haemodialysis because of PD technique failure. (B). Baseline dialysate CCL18 levels were, higher in patients who progressed to EPS group during one year follow-up.

FIG. 18. Dialysate Angiogenin levels in one year follow up. (A) Dialysate Angiogenin as a predictor of technique failure. Higher dialysate Angiogenin levels were predictive of switching to haemodialysis because of PD technique failure in one year follow-up. (B) Dialysate Angiogenin as a predictor of EPS. Patients developed EPS during one year follow up had higher dialysate Angiogenin in the preceding year. However, the difference was not statistically significant.

### EXAMPLE 1

### Detection of Novel Mediators in Renal Disease

In order to screen for mediators of inflammation and fibrosis in renal disease, the following study was performed using a discovery approach using an antibody array. Then, the identified mediators were quantified by specific ELISA.

### General Methods

CCL18 and angiogenin were measured in biological samples from patients with renal failure. We separated cells from the serum and spent peritoneal dialysate by centrifugation. CCL18 and angiogenin were detected by specific immunoassays. This includes antibody arrays and captured ELISA.

### Antibody Arrays

### Antibody Array

An antibody array (Antibody Array VI and VII) which can detect 120 proteins (Raybio®; Raybiotech Inc, 150 Technology Parkway, Norcross, Ga. 30092, USA) simultaneously was used. Although the antibody arrays are commercially available, their application has not been reported in dialysate before. Dialysates collected at the end of 4 hours PET were analysed. The positive signals were detected by autoradiography, as described below. From our previous work we have found that monocyte chemoattractant protein-1 (MCP-1) was present in the dialysate of PD patients ⁶. We were interested in mediators that were present in similar or even higher concentration as MCP-1 in the dialysate.

Arrays membranes were incubated with 2 mls of blocking buffer for 30 minutes. Blocking buffer was decanted. Then, the spent peritoneal dialysates were added and incubated for 2 hours. Arrays were washed with wash buffer I (supplied with the array)×three times with 5 minutes shaking on each wash. This was followed by wash×2 by wash buffer II (supplied with the array). Diluted biotin conjugated antibodies (supplied with the array) were added, 1 ml to each membrane. This was incubated for 2 hours or left overnight at 4° C. Wash step with both wash buffers were repeated again. 2 mls of 1000 fold diluted conjugated streptavidin was added to each membrane and incubated for 2 hours. Wash step was repeated again and detection buffers were added and incubated for 2 minutes at room temperature. Excess buffer was gently drained and array placed between 2 plastic covers provided. Membranes were exposed to Kodak x-omat film at room temperature.

### ELISA

Based on the antibody array study, we used specific ELISA to investigate CCL18 and angiogenin. Captured ELISA was set up for both molecules. Recombinant protein standards and matched paired antibodies were purchased from R & D Systems, Oxon, UK. Although these protein standards and matched paired antibodies were commercially available, their application has not been reported in dialysate before.

### Recruitment of Patients

The proposal has been approved by the local research ethics committee. We have recruited 106 patients (from Charing Cross and Hammersmith Hospital) since 2002. Serial serum and dialysate samples from these patients have been aliquoted and stored at −80° C. freezer. Demographic data, including dialysis regime, and medication history is collected and stored in an anonymous coded database.

### Peritoneal Membrane Transport Assessment and Sample Collection

Peritoneal membrane transport was measured using standard 4 hour peritoneal equilibrium test (PET) 9 at time of recruitment into the study (time=0), and then at 12 and 24 months. Patients were free from infection for at least 3 months prior to entry into the study. All patients use 2.27% dextrose dialysate overnight prior to PET. Blood samples are collected 2 hours following initiation of PET for serum biochemistry including C-reactive protein; aliquots of serum are: stored at −80° C. for measurement of novel mediators. After centrifugation, supernatants of 4 hours timed collection during the PET are aliquoted and stored at −80° C. for subsequent measurement of mediators.

### Results

### Discovery Using Antibody Array

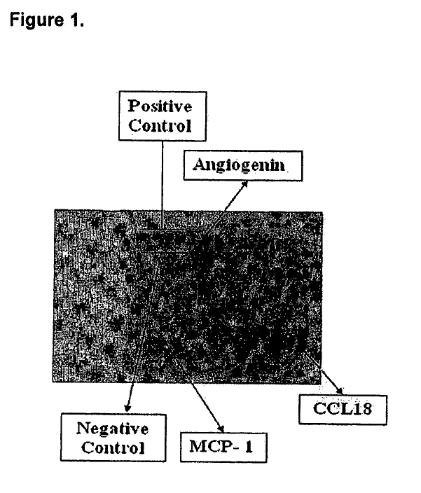

Using antibody array, we detected high concentrations of several novel proteins in the spent dialysate of 17 PD patients free from bacterial peritonitis for at least 3 months. We found that expression of CC chemokines in PD is very selective: i.e. high concentrations of CCL18 and MCP-1 were detected, but only low concentrations of MCP-2, MCP-3, MCP-4 and RANTES were present ¹⁰ (FIG. 1). We also found very high concentrations of angiogenin in the spent dialysate from PD patients (FIG. 1). To our knowledge, this is the first report of detection of CCL18 and angiogenin in PD.

### Quantification of Novel Mediator Using ELISA and Relationship to Clinical Situation

We measured the concentration of CCL18 and angiogenin in the dialysate and serum of 101, increased to 106, patients on long term PD.

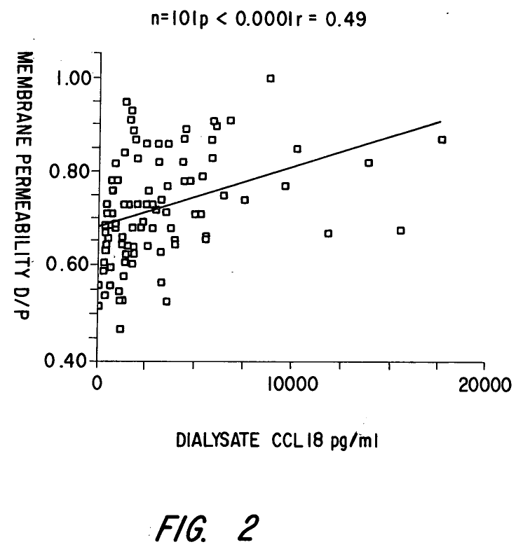

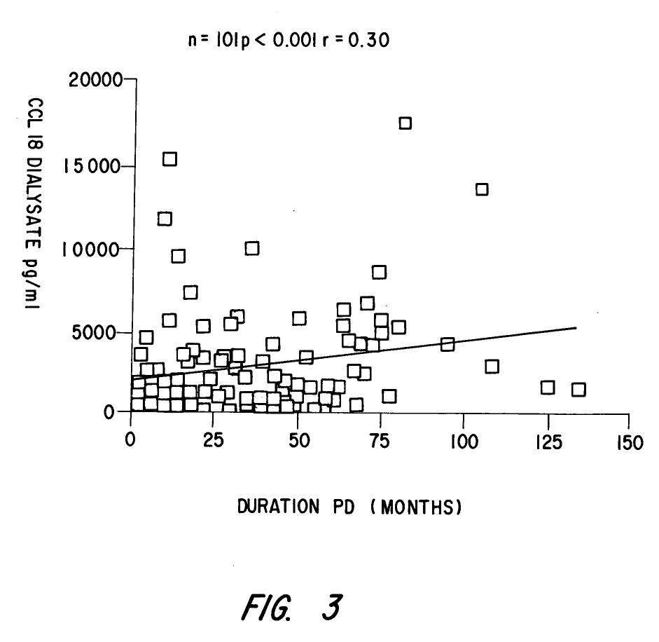

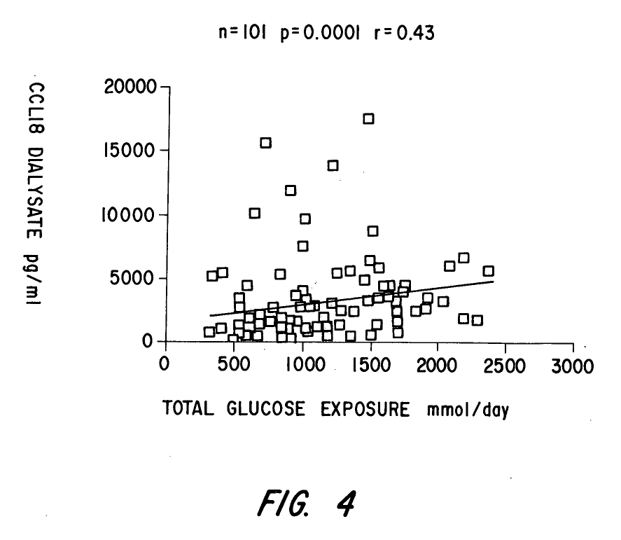

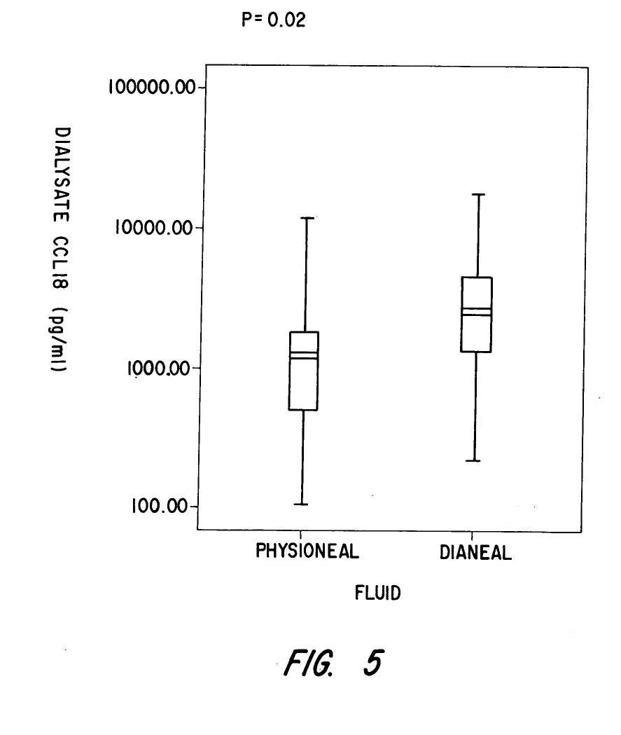

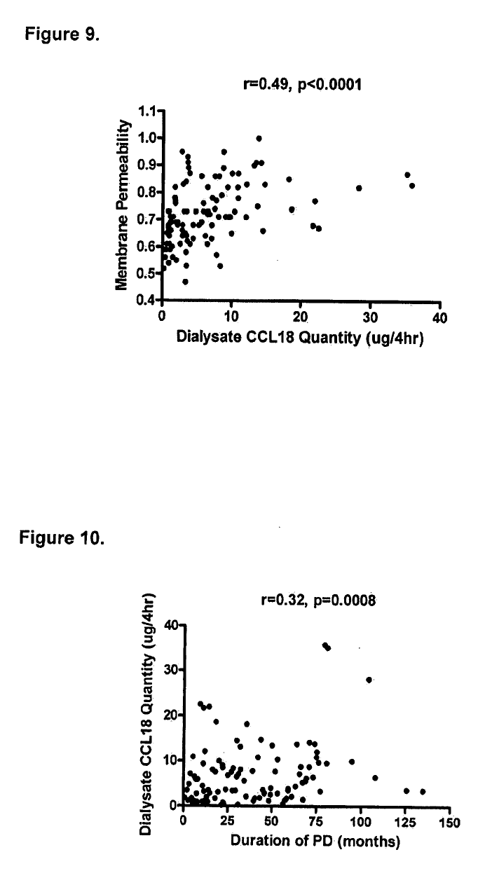

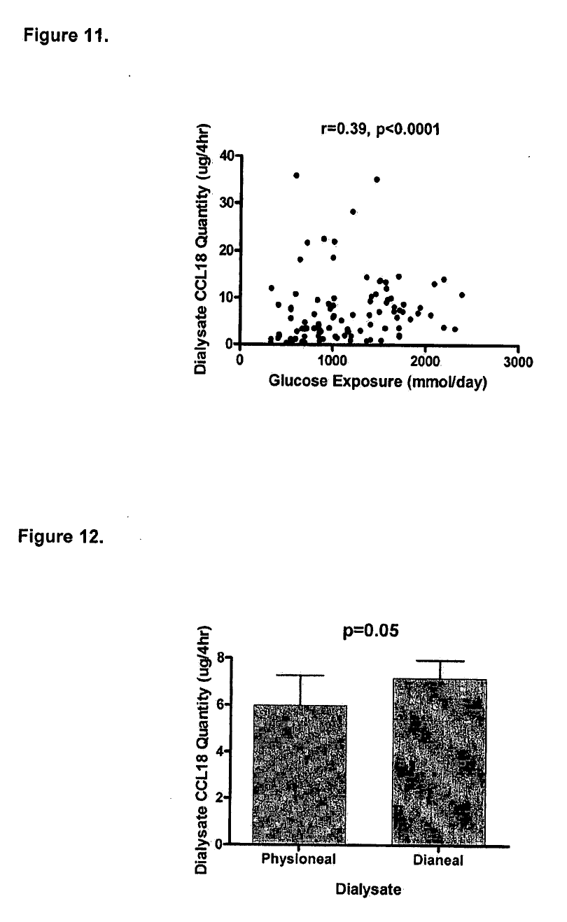

We found that dialysate CCL18 (quantity over 4 hour collection) correlated with peritoneal membrane permeability (dialysate/plasma (D/P) creatinine) in a standard 4 hour PET (FIGS. 2 and 9). We also found that dialysate CCL18 correlated with clinical risk factors that may be associated with chronic inflammation and fibrosis; including time on PD, bioincompatibility of dialysate and dose of glucose used in dialysis (FIGS. 3-5, 9 to 12).

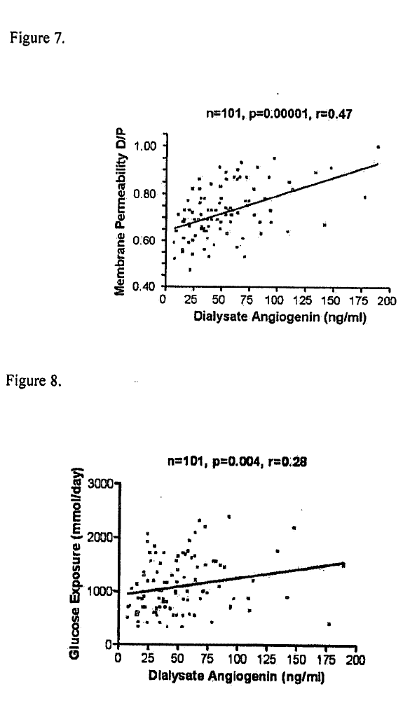

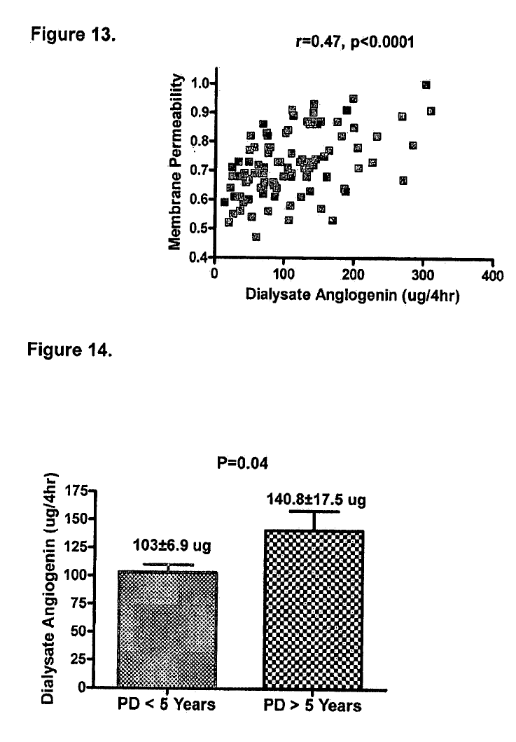

Based on analysis of samples from 98 patients so far, dialysate angiogenin correlated with membrane permeability (r=47, p<0.0001), (FIGS. 7 and 13).

### Discussion

Using a multiplex analysis of spent peritoneal dialysate from long term PD patients, we have discovered two mediators of peritoneal membrane dysfunction (CCL18 and angiogenin). The presence and importance of CCL18 and angiogenin have not been reported in renal diseases or complications of dialysis before. Initially, these two mediators were studied further by ELISA because of their relative high concentrations. Subsequently, we discovered that the concentration of CCL18 and angiogenin correlated with physiological measurement of peritoneal membrane dysfunction and clinical factors that are relevant to complications in long term PD.

CCL18, was originally discovered and named as pulmonary and activation-regulated chemokine (PARC), dendritic cell-chemokine-1 (DC-CK1), alternate macrophage activation-associated CC chemokine-1 (AMAC-1) and macrophage inflammatory protein-4 (MIP-4), and is a chemokine for T cells ¹¹. High concentrations of CCL18 have been detected in lung tissue, bronchoalveolar lavage cells and fluid, and serum of patients with lung fibrosis 12, Overexpression of CCL18 in murine lung results in T cell infiltration and lung fibrosis. CCL18 induced fibroblast synthesis of collagen both directly independent of TGF-13, and indirectly by recruiting pro-fibrotic lymphocytes¹². However, the importance of CCL18 has never been considered in renal diseases or complications of dialysis. In our study, we found that CCL18 correlated with physiological measurement of peritoneal membrane dysfunction and clinical risk factors which may lead to peritoneal membrane injury in PD patients.

Angiogenin is a 14.4 KDa protein, one of most potent angiogenesis factors 14. It has a structural similarity to Ribonuclease A. Cells that express angiogenin include vascular endothelial and smooth muscle cells, fibroblasts, normal colonic epithelium, normal peripheral blood lymphocytes, lung and colonic epithelial tumour cell lines, and primary gastrointestinal adenocarcinomas 15. The possible role of angiogenin in complications of peritoneal dialysis has not been studied. In our study, we demonstrated that dialysate angiogenin correlated with physiological: measurement of peritoneal membrane dysfunction and the amounts of glucose exposure during regular dialysis.

### EXAMPLE 2

### Results with Serum Measurement

Serum CCL18 concentrations correlated with membrane permeability (r=0.43, p<0.0001), the duration on PD (r=0.54, p<0.0001), amount of glucose used in PD (r=0.30, p=0.001), bioincompatibility fluid usage (p=0.001) previous episodes of peritonitis (r=0.28, p<0.005) and C reactive protein concentration (r=0.33, p<0.0001).

Serum angiogenin measurement in 77 patients did not show significant correlations with these clinical factors.

### EXAMPLE 3

### West London Prospective PD Study

A prospective observational study of the relationship between cytokines and clinical outcome in patients on PD was carried out with a total cohort of 106 patients from 2003-2007. Samples were collected from Charing Cross Hospital and Hammersmith Hospital PD patients if they were free from infection for 3 months prior to study. Informed written consent was obtained for the study.

Spent dialysate and serum samples were collected. Clinical data was also collected. Peritoneal membrane function was assessed by a clinical physiological test (PET Test and Adequacy). Cytokine Analysis was carried out with Multiplex and ELISA. All samples were taken at the time of standard Peritoneal Equilibrium Test. The 2 Hour Serum and 4 hour PD fluid samples were kept on ice, centrifuged at 3000 rpm for 15 minutes and stored at −80° C.

### EXAMPLE 4

### CCL18 May Predict Onset of Encapsulating Peritoneal Sclerosis (EPS): Evidence from West London Prospective PD Study.

We hypothesize that CCL-18 mediates peritoneal inflammation and fibrosis resulting in ultrafiltration failure and EPS.

### Methods

106 PD patients; free from acute/recent peritonitis were studied. Serum and dialysate samples taken at standard peritoneal equilibrium test measuring membrane permeability (MP). CCL-18 was measured by standard ELISA (R&D Systems) and correlated with clinical parameters.

### Results

By multiple regression analysis, baseline serum CCL18 increases with raised MP (p<0.001) and duration of PD (p=0.001) (Table 1).

By multivariant analysis, the quantity of CCL18 in dialysate (6.76±0.66 μg/4 h) increases with increasing MP (p<0.0001) and total glucose exposure/day (p<0.05) (Table 2), but is not related to type of dialysate, duration of PD, local and systemic inflammation (CRP, past peritonitis) or residual renal function.

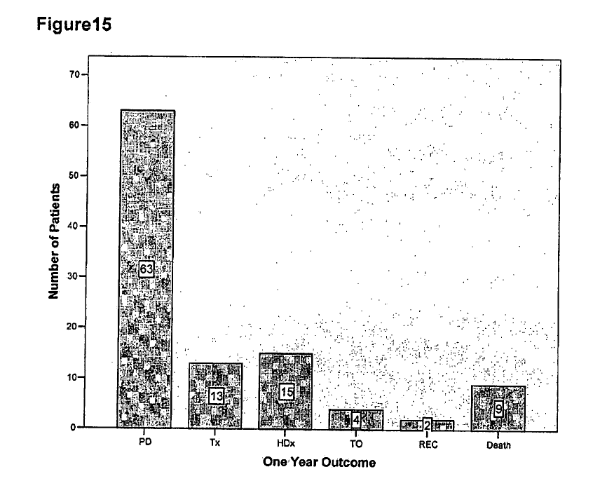

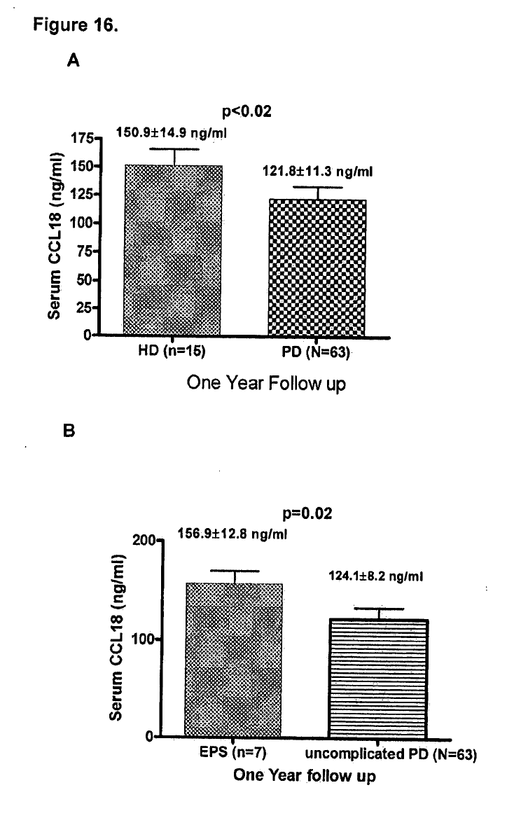

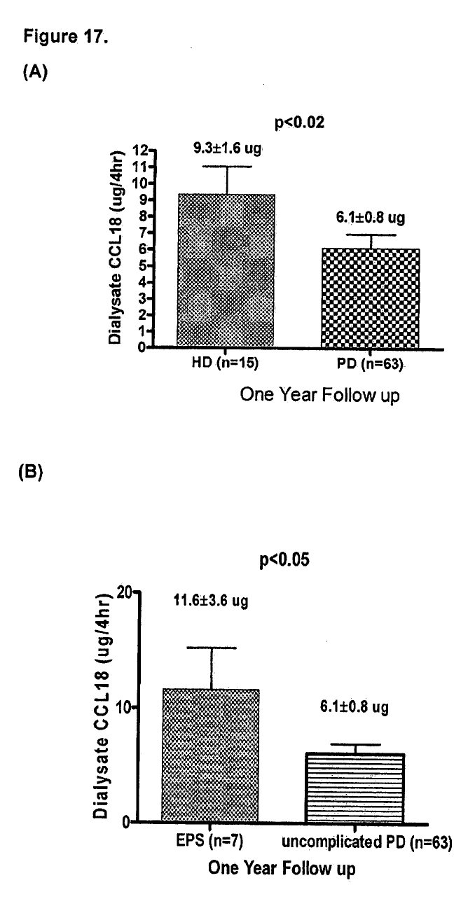

During 12 months follow-up, 15 patients transferred to haemodialysis (HD) 7 patients developed EPS, 13 had successful transplantation, 9 patients have died, 4 patients were transferred to other hospitals and 2 patients have recovered renal function (FIG. 15). Baseline serum CCL18 is significantly higher in group who transferred to HD (150.9±14.9 ng/ml, p=0.0:1) within 12 months of prospective follow up (FIG. 16A), and in patients who developed EPS (156.9±12.8 ng/ml, p=0.02) (FIG. 16B) in comparison to patients who remain on PD without complications (121.8±11.3 pg/ml) (FIG. 16A). Dialysate CCL18 showed a similar trend for higher quantity in patients who transfer to HD (9.3±1.6 μg/4 h, p=0.02) (FIG. 17A), developed EPS (11.6±3.6 μg/4 h, p<0.05) (FIG. 17B) in comparison to uncomplicated patients (6.1±0.8 μg/4 h) (FIG. 17).

Different clinical perimeters may be inter-related. For example, patients treated with APD often had been on PD for longer duration, lower residual renal function and had higher membrane permeability (MP) than CAPD patients, therefore APD patients need to use a larger volume of dialysate daily and consequently had higher glucose exposure. A range of factors may contribute to inflammation and contribute to endothelial dysfunction, such as smoking, past peritonitis and biocompatibility of dialysate (Physioneal vs Dianel). CRP is a systemic marker of inflammation. Therefore, the following candidate predictor variables for multivariate analysis: Modality (CAPD & APD); Age; Membrane Permeability (MP); CRP; Corrected Residuals (residual renal function); Glucose Exposure; Past Peritonitis (#episodes); Fluid (physioneal & dianeal); Smoking (smoker, non-smoker & ex-smoker); and Logarithm of duration of PD.

TABLE 1
Multivariate Analysis: Serum CCL18 Concentration

| Parameter | B | SEM | p |
|---|---|---|---|
| MP | 0.726 | 0.192 | <0.001 |
| Duration of PD (Log) | 0.170 | 0.049 | 0.001 |

CCL18 serum level increases with increasing MP and increasing duration of PD.

TABLE 2
Multivariate Analysis: Dialysate CCL18 (μg/4 hr)

| Parameter | B | SEM | p |
|---|---|---|---|
| MP | 1.653 | 0.397 | <0.001 |
| Glucose exposure mmol/day | 0.00019 | 0.000009 | 0.033 |

Increased dialysate CCL18 correlated with increasing MP and glucose exposure from dialysate.

### Discussion

Using multivariate analysis, both serum and dialysate CCL18 is higher with high membrane permeability. Serum CCL18 levels correlated with the duration of dialysis. Glucose exposure from dialyate also correlated with serum CCL18.

The prognostic value of CCL18 measurement was assessed with a one year follow up. Both serum and dialysate CCL18 levels are significantly higher in the groups who transferred to haemodialysis or developed EPS at one year follow-up. Therefore, serum and dialysate CCL18 may be used as a non-invasive prognostic marker of fibrosis/technique failure on PD.

### EXAMPLE 5

### Angiogenin can Predict Technique Failure in Long Term PD Patients: Evidence from West London Prospective PD Study.

Ultrafiltration failure with altered membrane transport requiring transfer to HD is a major complication of long-term PD. The exact pathophysiology is not fully understood. The potential role of angiogenin, a powerful angiogenic factor, has not been studied in PD patients.

We hypothesize that dialysate Angiogenin would predict ultrafiltration problems and technique failure in PD: patients.

### Methods

Prospective cohort study of 99 PD patients; all were free from acute/recent peritonitis at time of study. Samples taken at standard peritoneal equilibrium test were centrifuged and stored at −80° C. The presence of angiogenin in dialysate was discovered using multiplex antibody array (Raybiotech) and then quantified using specific ELISA (R&D Systems) and correlated with clinical parameters.

### Results

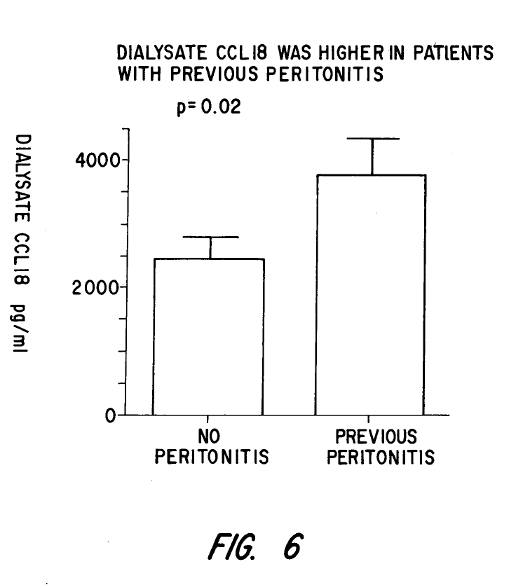

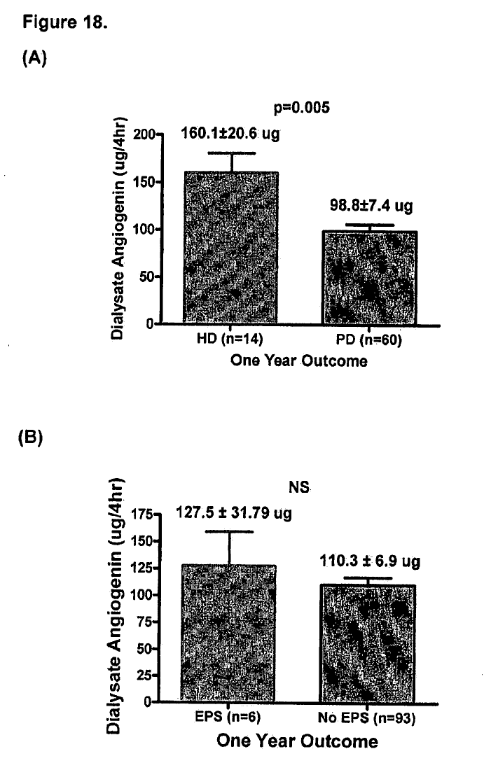

At entry of the study, dialysate Angiogenin correlated with membrane permeability (r=0.47 and p<0.0001) (FIG. 13). 21 of the 99 patients had been on PD for more than 5 years. Quantity of dialysate Angiogenin was significantly higher in patients on PD for ≧5 years (140.8±17.5 vs. 103±6.9 μg, p=0.04) (FIG. 14). After 12 months follow up, 14 patients had transferred to HD (duration of PD 4.8±0.7, range 1.49-10.4 years) (FIG. 18A) and 6 patients developed EPS (duration of PD 6.2±1.02 years, range 2.66-8.99 years) (FIG. 18B). Baseline dialysate angiogenin quantity was significantly higher in those who transferred to HD (160.1±20.6 vs. 98.8±7.4 μg, p=0.005) (FIG. 18A). Dialysate angiogenin quantity was also higher in patients who developed EPS one year later, however it did not reach significance (p=0.54) (FIG. 18B).

### Conclusions

To our knowledge, this is only the study that has demonstrated the presence and the prognostic value of angiogenin in the spent dialysate from PD patients. Dialysate Angiogenin levels correlated with membrane permeability. Also, patients who were transferred to haemodialysis at the end of one year had higher baseline dialysate Angiogenin levels.

The association of high angiogenin levels with future transfer to HD suggests that neo-angiogenesis is a key factor in the pathogenesis of changes in the peritoneal membrane with time on PD. An additional fibrotic component may be needed to provoke the development of EPS.

### REFERENCE LIST

1. Davies S J, Phillips L, Griffiths A M, Russell L H, Naish P F, Russell G I: What really happens to people on long-term peritoneal dialysis? Kidney Int 54:2207-2217, 1998
2. Davies S J, Bryan J. Phillips L, Russell G I: Longitudinal changes in peritoneal kinetics: the effects of peritoneal dialysis and peritonitis. Nephrol Dial Transplant 11:498-506, 1996
3. Margetts P J, Churchill D N: Acquired ultrafiltration dysfunction in peritoneal dialysis patients. J Am Soc Nephrol 13:2787-2794, 2002
4. Williams J D, Craig K J, Topley N, von Ruhland C, Fallon M, Newman G R, Mackenzie R K, Williams G T: Morphologic changes in the peritoneal membrane of patients with renal disease. J Am Soc Nephrol 13:470-479, 2002
5. McLoughlin R M, Witowski J, Robson R L, Wilkinson T S, Hurst S M, Williams A S, Williams J D; Rose-John S, Jones S A, Topley N: Interplay between IFN-gamma and IL-6 signaling governs neutrophil trafficking and apoptosis during acute inflammation. J Clin Invest 112:598-607, 2003
6. Malik A R, Little M A, Henriksson M, Tam F W, Brown E A: Peritonitis, peritoneal inflammation and membrane permeability: a longitudinal study of dialysate and serum MCP-1 in stable patients on peritoneal dialysis. J Nephrol 20:340-349, 2007
7. Szeto C C, Chow K M, Poon P. Szeto C Y, Wong T Y, Li P K: Genetic polymorphism of VEGF: Impact on longitudinal change of peritoneal transport and survival of peritoneal dialysis patients. Kidney Int 65:1947-1955, 2004
8. Zarrinkalam K H, Stanley J M, Gray J, Oliver N, Faull R J: Connective tissue growth factor and its regulation in the peritoneal cavity of peritoneal dialysis patients. Kidney Int 64:331-338, 2003
9. Twardowski Z J: Clinical value of standardized equilibration tests in CAPD patients. Blood Purif 7:95-108, 1989
10. Ahmad S, Bhangal G, Brown E A, Tam F W K: Novel cytokines are involved in complex regulation of inflammation in peritoneal dialysis [Abstract]. J Am Soc Nephrol 16:271A, 2005
11. Schutyser E, Richmond A, Van D J: Involvement of CC chemokine ligand 18 (CCL18) in normal and pathological processes. J Leukoc Biol 78:14-26, 2005
12. Luzina I G, Papadimitriou J C, Anderson R, Pochetuhen K, Atamas S P: Induction of prolonged infiltration of T lymphocytes and transient T lymphocyte-dependent collagen deposition in mouse lungs following adenoviral gene transfer of CCL18. Arthritis Rheum 54:2643-2655, 2006
13. Luzina I G, Tsymbalyuk N, Choi J, Hasday J D, Atamas S P: CCL18-stimulated upregulation of collagen production in lung fibroblasts requires Sp1 signaling and basal Smad3 activity. J Cell Physiol 206:221-228, 2006
14. Badet J: Angiogenin, a potent mediator of angiogenesis. Biological, biochemical and structural properties. Pathol Biol (Paris) 47:345-351, 1999
15. Pavel M E, Hassler G, Baum U, Hahn E G, Lohmann T, Schuppan D: Circulating levels of angiogenic cytokines can predict tumour progression and prognosis in neuroendocrine carcinomas. Clin Endocrinol (Oxf)62:434-443, 2005

1-11. (canceled)

12. A method for aiding in the assessment of peritoneal membrane injury or complications of peritoneal dialysis (PD) in a patient, the method comprising determining the level of CCL18 and/or angiogenin nucleic acid or protein in a sample from the patient.

13. The method of claim 12 wherein the sample is a blood serum sample, blood plasma sample or a sample of spent peritoneal dialysis fluid.

14. The method of claim 12 wherein the method is for assessing peritoneal disease activity in the patient.

15. The method of claim 13 wherein the method is for assessing peritoneal disease activity in the patient.

16. The method of claim 14 wherein the method is for assessing the likely progression of peritoneal membrane injury or complications of PD in the patient.

17. The method of claim 15 wherein the method is for assessing the likely progression of peritoneal membrane injury or complications of PD in the patient.

18. The method of claim 12 wherein the method is for assessing response of the patient to treatment.

19. The method of claim 13 wherein the method is for assessing response of the patient to treatment.

20. The method of claim 18 wherein the method is for assessing the likely progression of response of the patient to treatment.

21. The method of claim 19 wherein the method is for assessing the likely progression of response of the patient to treatment.

22. The method of claim 12 wherein the peritoneal membrane injury is a complication of peritoneal dialysis (PD).

23. The method of claim 22 wherein the complication of peritoneal dialysis is ultrafiltration failure (UFF) or encapsulating peritoneal sclerosis (EPS).

24. A method for assessing a peritoneal dialysis treatment regime or treatment regime for peritoneal membrane injury the method comprising the step of determining the level of CCL18 and/or angiogenin nucleic acid or protein in a sample from patients receiving the treatment regime.

25. A method for identifying a compound useful in peritoneal dialysis, for example in treating or preventing a complication of PD, or in treating or preventing peritoneal membrane injury, the method comprising the steps of a) determining whether a test compound is capable of suppressing production of, or activity of, CCL18 or angiogenin in peritoneal tissue or a sample from a patient with peritoneal membrane injury or receiving PD and b) selecting a compound which is capable of suppressing production of, or activity of, CCL18 or angiogenin in peritoneal tissue or in a patient with peritoneal membrane injury or receiving PD.

26. A method of treating a patient with peritoneal membrane injury or receiving PD, the method comprising the step of administering to the patient an effective amount of an antagonist of CCL18 and/or an antagonist of angiogenin.
